(19) United States
(12) Patent Application Publication
(10) Pub. No.: US 2023/0136733 A1
(43) Pub. Date: May 4, 2023

(54) PHYSICIAN-CONTROLLED HANDLE FOR MEDICAL DEVICES

(71) Applicant: Cook Medical Technologies LLC, Bloomington, IN (US)

(72) Inventors: Benjamin Cahill, Winston-Salem, NC (US); Meredith Lee, Winston-Salem, NC (US); Michael Entz, Winston-Salem, NC (US); Michael Williams, Winston-Salem, NC (US); Michael Brecht, Pfafftown, NC (US); Hunter Pelham, Winston-Salem, NC (US); Michelle Martinez, Winston-Salem, NC (US)

(21) Appl. No.: 17/976,364

(22) Filed: Oct. 28, 2022

Related U.S. Application Data
(60) Provisional application No. 63/273,724, filed on Oct. 29, 2021.

(51) Int. Cl. A61B 1/00; A61B 1/018; A61B 17/128

(52) U.S. Cl. CPC A61B 1/00133; A61B 1/018; A61B 17/1285

(57) ABSTRACT

A medical device includes a handle that includes a first member and a second member. Outer and inner elongate members of the medical device project from a distal end of the first member. The first and second members move, such as rotationally or pivotably move, relative to each other to control relative longitudinal movement of the outer and inner elongate members.

### CROSS-REFERENCE TO RELATED APPLICATIONS

This application claims the benefit of priority to U.S. Provisional Application No. 63/273,724, filed Oct. 29, 2021. The contents of U.S. Provisional Application No. 63/273,724 are incorporated by reference in their entirety.

### TECHNICAL FIELD

The present invention relates generally to medical devices, and more particularly to handles for endoscopic medical devices.

### BACKGROUND

Endoscopic medical devices often include an elongate tubular outer member and an elongate inner member movably disposed within the elongate tubular member. The distal end of the inner member includes a distal element (such as, a clip, forceps, or a snare) for performance of a surgical procedure within a patient. A handle is connected to proximal ends of the outer and inner members to control longitudinal and axial rotational movement of one member relative to the other, as well as operation of the distal element.

To perform a surgical procedure, the outer and inner members are advanced through a working channel of an endoscope until a distal end of the medical device is at a target treatment site within a patient. Once the distal end at the treatment site, the distal element is manipulated with the handle to perform a desired action on tissue at the treatment site.

The surgical procedure is typically performed with two people. One person, usually a physician, holds the endoscope with one hand and longitudinally advances and retracts the inner and outer members through the working channel by grasping the outer member with his/her other hand. A second person, usually a nurse technician, holds and controls the handle in order to control the relative movement of the inner and outer members and operation of the distal element. The second person controls the handle in accordance with instructions given orally by the first person.

More and more, physicians desire to control the movement of the inner and outer members and the distal element at the treatment site, rather than issue instructions to the nurse technician. However, when the distal end is at the treatment site, a length of the inner and outer members extending from the proximal opening of the endoscope to the handle is usually too great, such that the physician cannot effectively operate the handle while also holding the endoscope. As such, ways to provide the physician with control of the outer and inner members while also being able to hold the endoscope may be desirable.

### BRIEF SUMMARY

The present description describes medical devices that include a handle that an operator can hold and control with one hand. The handle may be operatively coupled to elongate inner and outer members that are configured to move relative to each other. In addition, the handle may be configured to allow an operator to hold the handle sufficiently close to an endoscope during a medical operation, including during times that the inner and outer members are extending within the endoscope to a treatment site within the patient, which in turn may allow the operator to simultaneously hold and control both the medical device and the endoscope. Accordingly, the handle embodiments described herein may provide an operator holding an endoscope with enhanced control of the medical device extending through the endoscope during an operation, compared to other handles that necessitate two operators to handle and control the medical device handle and the endoscope.

In one embodiment, a handle for a medical device includes: a first member from which an inner elongate member and an outer elongate member extend; and a second member configured to move toward and away from the first member, wherein movement of the second member toward and away from the first member controls relative longitudinal movement of the inner and outer elongate members.

In another embodiment, a medical device includes: an outer elongate member, an inner elongate member, a distal end effector coupled to a distal end of the elongate inner member, and a handle. The inner member is movably disposed within the outer elongate member, where each of the outer elongate member and the inner elongate member longitudinally extend from a proximal portion to a distal portion. The handle is coupled to the proximal portion, and includes a first member and a second member. The outer and inner elongate members project from a distal end of the first member. Additionally, the first and second members are configured to pivot relative to each other to longitudinally move the outer and inner elongate members relative to each other.

In another embodiment a method of operating a medical device includes: positioning a handle in an open state, wherein the handle comprises a first member and a second member pivotably connected to each other, wherein outer and inner elongate members of the medical device distally extend from a distal end of the first member, wherein, in the open state, the first and second members form an angle less than 90 degrees; rotating the second member toward the first member; and proximally moving the inner elongate member relative to the outer elongate member in response to rotating the second member toward the first member.

Other embodiments are possible, and each of the embodiments can be used alone or together in combination. Accordingly, various embodiments will now be described with reference to the attached drawings.

### BRIEF DESCRIPTION OF THE DRAWINGS

FIG. 1A shows a perspective view of a medical device including a handle in an open state.

FIG. 1B shows a perspective view of the medical device of FIG. 1A, with the handle in a closed state.

FIG. 2 shows a cross-sectional side view of the medical device of FIGS. 1A, 1B.

FIG. 3A shows a perspective view of the medical device of FIG. 1A-2 being operated by a hand of an operator, with the handle in an open state, and with a distal end effector configured as a clip assembly.

FIG. 3B shows a perspective view of the medical device of FIG. 3, but with the hand configuring the handle in a closed state.

FIG. 4 shows a perspective view of a medical system including an endoscope and the medical device of FIGS. 1A-3B.

### DETAILED DESCRIPTION

The present description describes various embodiments of a handle for a medical device, medical devices and medical systems that include a handle, and related methods that include operating a handle for controlling movement, including relative longitudinal movement and/or axial rotational movement, of inner and outer elongate members of a medical device and/or a distal end effector coupled to one or both of the elongate members. During an endoscopic medical procedure, the handle may be positioned relatively close to a proximal opening of a working channel of an endoscope after distal ends of the outer and inner members have reached a treatment site within a patient, such that an operator is able to handle both the endoscope and the handle.

Though use of the handle of the present description, an operator handling the endoscope, which is often a physician, can simultaneously control relative longitudinal and/or rotational movement of the outer and inner members and a distal member of the medical device used to perform an operation on tissue at a treatment site within the patient. In turn, the control of the movement of the outer and inner members does not have to depend on communication between a first operator controlling the endoscope and combined longitudinal movement of the outer and inner members and a second operator controlling relative longitudinal and rotational movement of the outer and inner members. This, in turn, may allow for quicker and more precise and accurate placement, movement, and/or deployment of the distal member at the treatment site. Various example embodiments of the handle are now described.

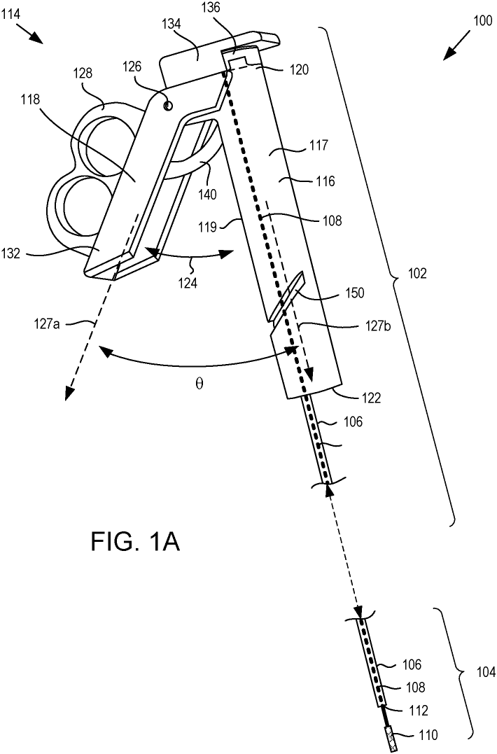

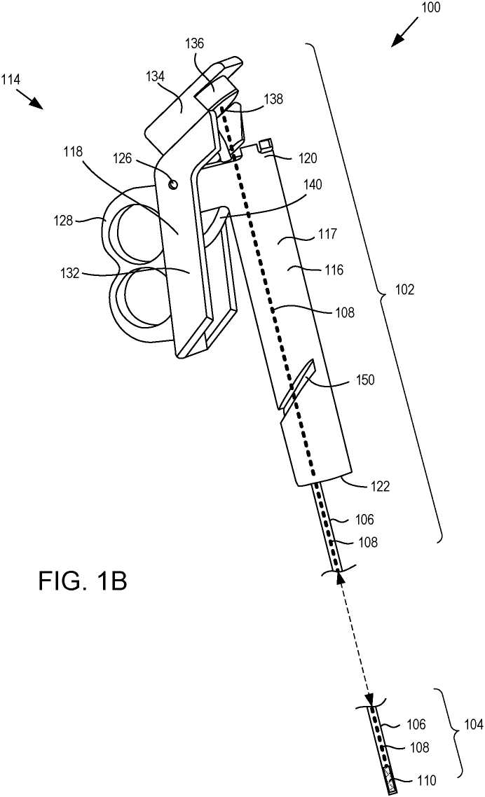

FIGS. 1A and 1B show a perspective view of an example embodiment of a medical device 100 extending from a proximal portion 102 to a distal portion 104. The medical device 100 includes an outer elongate member 106 and an inner elongate member 108, each extending from the proximal portion 102 to the distal portion 104. The inner elongate member 108 is movably disposed within (e.g., within a lumen of) the elongate outer member 106. The outer and inner elongate members 106, 108 may be configured to longitudinally move relative to each other. Such relative longitudinal movement is used or leveraged to perform a medical procedure. For example, the medical device 100 may further include a distal element 110 that is configured to perform an action on tissue at a treatment site within a patient associated with the medical procedure. Non-limiting examples of the distal element include a clip or clip assembly (e.g., a hemostatic clip or clip assembly used to achieve hemostasis at a bleeding site), forceps, an electrode or electrode assembly, a snare, a distal end of a needle knife, or a cutting edge (e.g., of a sphincterotome). The distal element 110 may be attached or connected to the inner member 108, such that longitudinal and rotational movement of the inner member 108 correspondingly controls longitudinal and rotational movement of the distal element 110. Accordingly, relative longitudinal movement of the outer and inner members 106, 108 may be used to extend the distal element 110 distally past a distal end 112 of the outer member 108 in order to expose the distal element 110 to the tissue at the treatment (FIG. 1A), and to proximally retract the distal element 110 to within the outer member 108 (FIG. 1B).

The medical device 100 may further include a handle 114 at the proximal portion 102 coupled to the outer and inner elongate members 106, 108, and that is configured to control relative longitudinal and/or axial rotational movement of the outer and inner members 106, 108. The handle 114 generally includes a pair of members movable relative to each other, including a first member 116 and a second member 118. As shown in FIGS. 1A, 1B, the outer and inner elongate members 106, 108 distally project or extend from the first member 116. For example, the first member 116 may include an elongate portion or body 117 that extends from a proximal end 120 to a distal end 122, and the outer and inner elongate members 106, 108 may project or extend from the distal end 122. The movement of the second member 118 may include movement toward and away from an elongate side 119 of the elongate body 117.

The relative movement between the first and second members 116, 118 may include two relative directions. In a first relative direction, the first and second members 116, 118 move toward or closer to each other. In a second relative direction, the first and second members 116, 118 move away from each other.

In addition, the relative movement between the first and second members 116, 118 may include or be characterized by the second member 118 being movable toward and away from the first member 116. For example, during a medical procedure, the first member 116, from which the outer and inner elongate members 106, 108 extend, is generally stable or static while the second member 118 moves toward and away from the first member 116. Correspondingly, movement of the first and second members 116, 118 toward each other may include the second member 118 moving toward the first member 116, and movement of the first and second members 116, 118 away from each other may include the second member 118 moving away from the first member 116.

In addition, for at least some embodiments such as shown in FIGS. 1A and 1B, the relative movement between the first and second members 116, 118 is a rotational or pivotable movement, as denoted by double-arrow 124 in FIG. 1A. The first and second members 116, 118 may be connected via a hinge 126 or other similar connection that provides a point, such as a pivot point, about which the first and second members 116, 118 rotationally and/or pivotably move relative to each other. For example, as shown in FIGS. 1A and 1B, the hinge 126 may be formed by a pin extending or projecting from the first member 116 perpendicular to the direction of the relative movement of the first and second members 116, 118, and one or more holes integrated with the second member 118 through which the pin extends. The pin and holes define a pivot point about which the first and second members relatively pivot or rotate. Ways for the first and second members 116, 118 to relatively pivot or rotate other than through use of a hinge may be possible.

Also, the relative movement of the first and second members 116, 118 may control relative longitudinal movement of the outer and inner elongate members 106, 108. The relative longitudinal movement may be performed in two opposing relative directions. In a first relative direction, the inner elongate member 108 distally moves relative to the outer elongate member 106, or the outer elongate member 106 moves proximally relative to the inner elongate member 108. In a second relative direction, the inner elongate member 108 moves proximally relative to the outer elongate member 106, or the outer elongate member 106 moves distally relative to the inner elongate member 108.

Further, relative movement of the first and second members 116, 118 in each of the relative directions may correspond to a respective one of the relative directions in which the outer and inner elongate members 106, 108 longitudinally move. In at least some embodiments such as shown in FIGS. 1A and 1B, relative movement of the first and second members 116, 118 toward each other may control or cause the inner elongate member 108 to proximally move relative to the outer elongate member 106, and relative movement of the first and second members 116, 118 away from each other may cause or control the inner elongate member 108 to distally move relative to the outer elongate member 106.

In addition, the handle 114 may be configurable in, and/or the first and second members 116, 118 may be configured to be in and to relatively move between, an open state or position and a closed state or position. FIG. 1A shows the handle 114 in the open state. FIG. 1B shows the handle 114 in the closed state. In the open state (FIG. 1A), the first and second members 116, 118 are positioned or disposed furthest apart from each other (e.g., they are unable to move further apart from each other). In the closed state (FIG. 1B), the first and second members 116, 118 are positioned or disposed closest to each other (e.g., they are unable to move closer to each other). The first and second members 116, 118 may also be configured in, positioned in, and/or moved to any of various intermediate states or positions, where the first and second members 116, 118 are capable of moving closer to each other or farther apart from each other.

As shown in FIG. 1A, in the open state, the first and second members 116, 118 may form an angle θ that is less than 90 degrees. FIG. 1A shows the angle θ as being defined by geometric rays 127a, 127b longitudinally extending over generally elongate profiles of the first and second members 116, 118, respectively. Additionally, the rays 127a, 127b generally extend away from the hinge 126 where the first and second members 116, 118 are connected, and toward the unconnected or free ends of the first and second members 116, 118, with the free end of the first member 116 being its distal end 122.

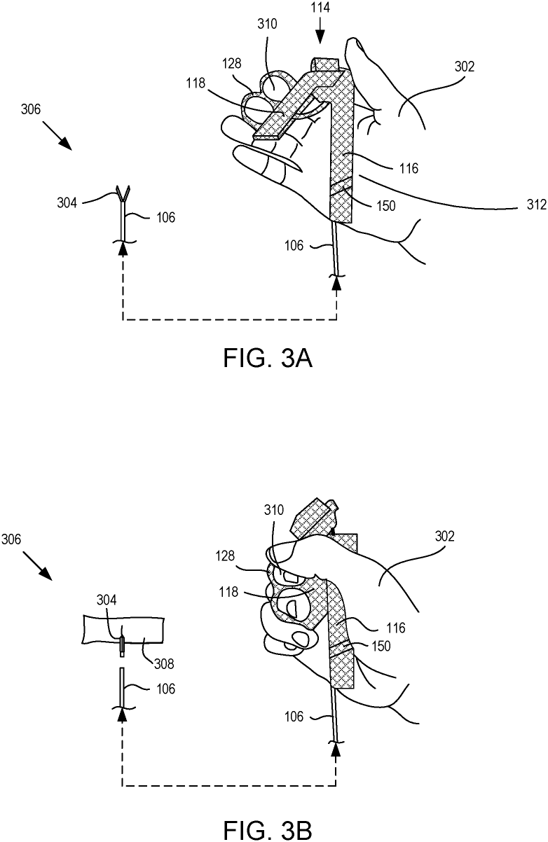

Additionally, as described in further detail below with respect to FIGS. 3A, 3B, the angle θ may be sufficiently small enough to allow an operator to grasp the first and second members 116, 118 with a generally open hand, and move the first and second members 116, 118 to the closed state (FIG. 1B) by generally closing the hand. At the same time, the angle θ may be sufficiently large to effect sufficient relative longitudinal movement between the outer and inner elongate members 106, 108. A

The second member 118 may include a finger-securing portion 128 that provides or defines an area through which an operator may insert and retain at least one finger of the hand used to operate the handle 114. In at least some embodiments as shown in FIGS. 1A, 1B, the finger-securing portion 128 may be in the form at least one finger ring, such as two finger rings, although other configurations, including those that define an area through which at least one finger may be inserted and/or retained though not in the shape of a ring, may be possible. Additionally, in at least some embodiments, the finger-securing portion 128 may project or extend from, and/or be disposed on, a surface of the second member 118 that faces in a direction that the second member 118 moves away from the first member 116. Accordingly, when secured by or retained within the finger-securing portion 128, the at least one finger may bias the second member 118 in a direction that moves the second member 118 toward the first member 116 to the closed state, and may bias the finger-securing portion 128 in a direction that moves the second member 118 away from the first member 116 to the open state.

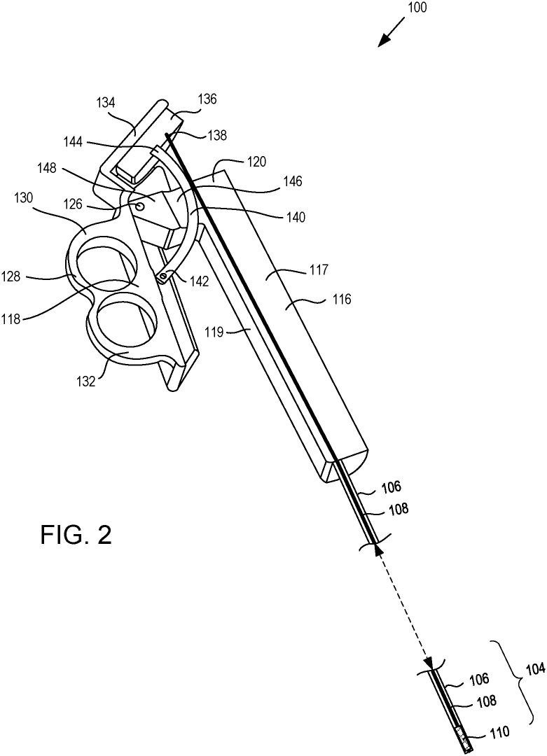

FIG. 2 shows cross-sectional side view of the handle 114, showing further structural and functional features of the handle 114 in more detail. As best shown in FIG. 2, for at least some embodiments, the second member 118 may include and/or be configured as a bell crank 130 that includes a pair of arms 132, 134 integrated with the hinge 126. The pair of arms 132, 134 may generally extend from and form or define an angle relative to the pivot point of the hinge 126. FIG. 2 shows the angle defined by the arms 132, 134 as being greater than 90 degrees and less than 180 degrees, although other angles may be possible.

As shown in FIG. 2, the first arm 132 includes the finger-securing portion 128. Also, the first arm 132 may be configured to move, such as pivot or rotate, toward and away from the elongate side 119 of the first member 116 as the handle 114 moves between closed and open states, respectively. For example, the first arm 132 may move toward the elongate side 119 as the handle 114 moves from the open state to the closed state, and may move away from the elongate side 119 as the handle moves from the closed state to the open state.

Additionally, the second arm 134 may be configured to engage and/or be positioned adjacent to the proximal end 120 of the first member 116 of the handle 114 when the handle 114 is in the open state, as shown in FIG. 1A. In addition, the second arm 134 may be configured to move, such as pivot or rotate, toward and away from the proximal end 120 of the first member 116 movement as the handle 114 moves between open and closed states, respectively. For example, the second arm 134 may move toward the proximal end 120 when the handle 114 moves from the closed state to the open state, and may move away from the proximal end 120 when the handle 114 moves from the open state to the closed state.

In addition, the second arm 134 may be connected and operably coupled to the inner elongate member 108. As shown in FIG. 1A-2, the inner elongate member 108 may proximally extend through and be movably disposed within the elongate body 117 of the first member. Through its connection to the inner elongate member 108, the second arm 134 may be configured to translate its movement, including pivotable movement about the pivot point of the hinge 126, to longitudinal movement of the inner elongate member 108 relative to the outer elongate member 108. For example, movement of the second arm 134 away from the proximal end 120 may proximally move the inner elongate member 108 relative to the outer elongate member 106, and movement of the second arm 134 toward the proximal end 120 may distally move the inner elongate member 108 relative to the outer elongate member 106.

In particular example embodiments, such as shown in FIG. 1A-2, the second arm 134 may include an inner member connecting portion 136 that fixedly attaches or connects to a proximal end 138 of the inner elongate member 108. Various way of fixedly attaching the distal end 138 of the inner elongate member 108 to the inner member connecting portion 136 may be possible. As one non-limiting example, the distal end 138 may extend at least partially through the inner member connecting portion 136, and a set screw may be positioned within and engaged with a threaded bore extending within the inner member connecting portion 136 in a direction perpendicular to a direction that the distal end 138 partially extends. The set screw may bias the distal end 138 against the inner member connecting portion 136 to fixedly attach the distal end 138 to the inner member connecting portion 136. Various other ways to fixedly attach the distal end 138 to the inner member connecting portion 136 may be possible.

Also, by being configured as a bell crank 130, the rotational or pivotable movement of the first and second arms 132, 134 relative to the first member 116 of the handle 114 may be opposite from each other. That is, when the handle 114 is in the open state such that the first arm 132 is in a position furthest away from the first member 116, the second arm 134 may be in a closest position to the proximal end 120 of the first member 116, as shown in FIG. 1A. In addition, when the handle 114 is in the closed state such that the second arm 134 is in a position closest to the first member 116, the second arm 134 may be in a position furthest away from the proximal end 120 of the first member 116, as shown in FIGS. 1A and 2. Correspondingly, when the first arm 132 moves toward the first member 116, the second arm 134 moves away from the proximal end 120, and when the first arm 132 moves away from the first member 116, the second arm moves toward the proximal end 120.

In sum, movement of the handle 114 from the open state to the closed state moves the second member 118 toward the first member 116, which includes movement of the first arm 132 pivoting toward the first member 116 and the second arm 134 pivoting away from the first member 116, causing proximal movement of the inner elongate member 108 relative to the outer elongate member 106. In addition, movement of the handle 114 from the closed state to the open state moves the second member 118 away from the first member 116, which includes movement of the first arm 132 pivoting away from the first member 116 and the second arm 134 pivoting ward the first member 116, causing distal movement of the inner elongate member 108 relative to the outer elongate member 106.

Additionally, for at least some embodiments such as shown in FIG. 1A-2, the second member 116 and/or the bell crank 134, may include an arm connection element 140 connected to the first and second arms 132, 134 that may provide structural support for the bell crank 130 and/or facilitate the pivotable movement of the first and second arms 132, 134. As shown in FIG. 2, the arm connection element 140 may circumferentially extend about the pivot point of the hinge 126 between the first and second arms 132, 134, and have a first end 142 that connects to the first arm 132 and a second end 144 that connects to the second arm 134. In particular embodiments such as in FIG. 2, the second end 144 connects to the inner member connection portion 138 of the second arm 134. Also, as shown in FIG. 2, the arm connection element 140 may extend through an area of the angle defined by the arms 132, 134 that is greater than 90 degrees and less than 180 degrees.

Through its connections to the arms 132, 134, the arm connection element 140 may move or pivot about the pivot point of the hinge 126 in the same direction as the movement of the arms 132, 134. For example, the first end 142 may follow movement of the first arm 132 toward and away from the first member 116, and the second end 144 may follow movement of the second arm 134 toward and away from the proximal end 120 of the first member 116.

Additionally, through its connections to the arms 132, 134, the arm connection element 140 may be configured to translate at least some force or bias on the first arm 132 to the second arm 134 to the corresponding rotational or pivotable movement of the arms 132, 134. For example, when an operator exerts a force on the first arm 132 to move the first arm 132 toward the first member 116, the arm connection element 140 may translate at least a portion of the force to the second arm 134 to facilitate movement of, such as by pushing, the second arm 134 away from the proximal end 120. Similarly, when an operator exerts a force on the first arm 132 to move the first arm 132 away from the first member 116, the arm connection element may translate at least a portion of the force to the second arm 134 to facilitate movement of, such as by pulling, the second arm 134 toward the proximal end 120. As shown in FIG. 2, the ends 142, 144 are connected to their respective arms 132, 134 at connection points each about half the lengths of the arms 132, 134, providing sufficient distances away from the pivot point to, in turn, provide an amount of torque that enhances the rotational or pivotable movement of the arms 132, 134.

In addition, for at least some embodiments as shown in FIG. 2, the arm connection element 140 may extend through and be configured to move through the first member 116. The first member 116 may include a groove or track 146 through which the arm connection element 140 extends. The track 146 may guide movement of the arm connection element 140 through the first member 116 and/or reduce friction between the arm connection element 140 and the first member 116 as the arm connection element 140 moves through the first member 116. For at least some embodiments as shown in FIG. 2, the first member 116 may be in the form of an L-shape or other similar shape formed by the elongate portion 117 and a second portion 148 that includes an arm or projection that extends or projects from, and is connected to, the elongate portion 117 at the proximal end 120. The hinge 126, which provides the pivot point for the bell crank 130, may be integrated with the second portion 148. For example, the hole for the hinge 126 may extend through the second portion 148. As shown in FIG. 2, the track 146 and the arm connection element 140 may extend through the second portion 146 between the hinge 126 and where the second portion and the elongate portion 117 connect.

FIGS. 3A and 3B show a method of operation of the handle 114 of the medical device 100 of FIG. 1A-2. The handle 114 may be sized and shaped such that an operator can grasp and optimally operate the handle with a single hand 302. FIG. 3A shows the operator’s hand 302 engaging with the handle 114 in the open state, and FIG. 3B shows the operator’s hand 302 engaging with the handle 114 in the closed state.

As a non-limiting example to illustrate the method, the device 100 is configured as a clip device, such as a hemostatic clip device, where the distal end effector 110 shown in FIG. 1A-2 is configured as a clip or clip assembly 304 that is deployed at a treatment site 306 to bind or join together tissue 308 at the treatment site 304 to stop bleeding, such as bleeding caused by removal of a polyp or a bleeding ulcer in the gastrointestinal (GI) tract, as non-limiting examples. However, the handle 114 may be generally used for any of various medical device embodiments to effect relative longitudinal movement of two elongate members 106, 108 and/or use any of various configurations of an end effector 110, as previously described.

As shown FIGS. 3A and 3B, the operator operates the handle 114 by simultaneously engaging with or contacting the first and second members 116, 118. In particular, when the operator has at least one of his/her fingers 310 inserted through or in the finger securing portion 128, the first member 116, including the elongate body 117, is positioned against the palm 312 of the operator’s hand 302. In some embodiments, the handle 114 is self-biased in the open state and/or configure or position itself in the open state absent external forces acting on it, such as by including a spring or other elastic member integrated with the first and/or second members 116, 118 that is configured to bias the first and second members 116, 118 away from each other in order to move or pivot the first and second members 116, 118 away from each other. Accordingly, the operator can open his/her hand 302 and/or uncurl his/her fingers 310 as shown in FIG. 3A, and the second member 118 will follow the movement of the fingers 304 and move away from the first member 116 due to the biasing from spring member. In other embodiments, the handle 114 does not have any self-biasing components, and configuring or positioning the handle 114 in the open state may be caused by external forces such as from the operator’s hand 302. In such latter embodiments, for example, movement of the first and second members 116, 118 away from each other may be caused, including solely caused, by the operator’s fingers 310 biasing the finger securing portion 128 when opening the hand 302. Additionally, to move the handle 114 from the open state (FIG. 3A) to the closed state (FIG. 3B), the operator may close his/her hand 302, such as if the operator is trying to form a fist while holding the handle 114 by curling or otherwise moving his/her fingers 310 toward the palm 312, which exerts a bias on the second member 118 that moves the second member 118 toward the first member 116.

Accordingly, when the operator wants to distally move the inner elongate member 108 relative to the outer elongate member 106, the operator can open his/her hand 302 to cause the second member 118 to move or pivot away from the first member 116 until the handle 114 is in the open state. The operator may distally move the inner elongate member 106 to effect a certain configuration of the distal end effector 110. For example, FIG. 3A shows that the operator opening his/her hand to move the handle 114 into the open state causes the clip assembly 304 to distally advance past a distal end of the outer elongate member 106 and the legs or jaws of the clip assembly 304 to open.

Additionally, when the operator wants so proximally move the inner elongate member 108 relative to the outer elongate member 106, the operator can close his/her hand 302 to cause the second member 118 to move or pivot toward the first member 116 until the handle 114 is in the closed state. Similarly, the operator may proximally move the inner elongate member 106 to effect a certain configuration of the distal end effector 110. For example, FIG. 3B shows that the operator closing his/her hand to move the handle 114 toward the closed state causes the legs or jaws of the clip assembly 306 to close to grasp the tissue 308, which in turn joins portions of the tissue 308 together to stop bleeding. Further closing his/her hand to move the handle 114 to the closed state may cause further proximal movement of the inner elongate member 108 relative to the outer elongate member 106, which in turn may cause the clip assembly 304 to separate from the inner elongate member 108 (and from the rest of the clip device), fully deploying the clip assembly 304 at the treatment site.

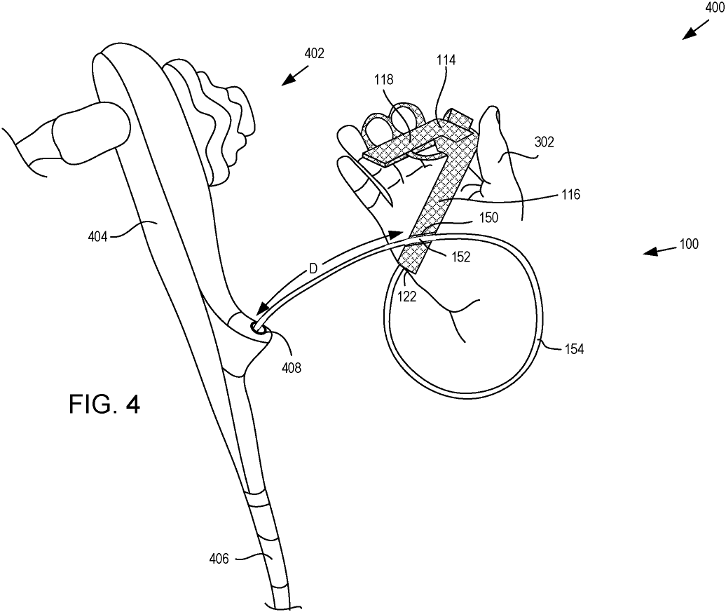

FIG. 4 shows a medical system 400 that includes the medical device 100 with the handle 114 in combination and used with an endoscope 402. In general, the endoscope 402 is used, among other things, to guide or facilitate the movement of the distal portion 104 (FIG. 1A-2) of the medical device 100 to a treatment site within a patient. The endoscope 402 includes a handle 404 and an insertion tube 406 that is inserted into a patient. The insertion tube 406 extends from a proximal end connected to the handle 404 to a distal end (not shown). The insertion tube 406 has a longitudinal length sufficient to reach the treatment site within the patient. The endoscope 402 further includes a working channel longitudinally extending through the insertion tube 406 and that proximally extends to a proximal opening 408 at a portion of the handle 404.

An operator, such as a physician, may insert the insertion tube 406 into an opening of a patient and distally advance the insertion tube 406 within the patient until a distal end of the insertion tube 406 reaches a desired location at or near the treatment site. The operator may then insert the distal portion 104 of the medical device 100 into the working channel proximal opening 408, and distally advance the outer and inner elongate members 106, 108 through the working channel until the distal portion 104 reaches the treatment site. The operator may do so by grasping and distally pushing the outer elongate member 106 through the working channel. While the operator is distally advancing the outer elongate member 106 with one hand, the operator may be simultaneously holding the endoscope handle 402 with his/her other hand.

For at least some operations using the medical system 400, while the operator is holding the endoscope 402 and distally advancing the distal portion 104 to the treatment site, a second operator, such as an assistant or nurse of the physician, may be holding the handle 114 of the medical device 100. As shown in FIG. 4, when the first operator has finished distally advancing the distal portion 104 to the treatment site, the handle 114 may be positioned or held a distance D from the working channel proximal opening 408 that is short enough that the first operator can simultaneously hold and control both the endoscope handle 404 and the device handle 114. As shown in FIG. 4, the device handle 114 may be held a sufficiently short distance D from the working channel opening 408 without the portions of the outer and inner elongate members 106, 108 between the first member 116 and the working channel opening 408 being too long so as to be kinked, too coiled or twisted or otherwise too tortuous so as to impeded or hinder their unison and/or relative longitudinal movement and operation of the handle 114.

Accordingly, when the first operator has finished advancing the distal portion 104 to the treatment site, the first operator may obtain the handle 114 from the second operator, such that the first operator is simultaneously holding the endoscope handle 404 and the medical device handle 114. At this time, the first operator has simultaneous control of the endoscope 402, including operation of the endoscope handle 404, and the medical device 100, including operation of the device handle 114. As a result of the simultaneous control, the first operator can control operation of the medical device 100 as desired while holding and controlling the endoscope 402. For example, while holding the endoscope 402, the operator, through control of the device handle 114, can control relative longitudinal movement of the outer and inner elongate members 106, 108, and/or can control operation of the distal end effector 110, such as operation and deployment of the clip assembly 304 shown in FIG. 3, as a non-limiting example.

Additionally, when the distal portion 104 has reached the treatment site and the operator is holding the endoscope 402 with one hand and holding the device handle 114 with his/her other hand the distance D from the working channel opening 408, a length of portions of the outer and inner elongate members 106, 108 between device handle 114 and the working channel opening 408 is short enough such that the first operator can effect unison longitudinal movement of the outer and inner elongate members 106, 108 and the distal end effector 110, through movement of the handle 114 toward and away from the working channel opening 408, without kinking or other similar undesirable interference of the outer and inner elongate members 106, 108 that may otherwise occur if the length of the outer and inner elongate members 106, 108 between the opening 408 and the handle 114 is too long.

The first operator can also move, such as by rotating, the handle 114 to effect axial rotational movement of the outer elongate member 106, the inner elongate member 108, and/or the distal end effector 110. In various embodiments, such movement of the handle 114 to effect axial rotational movement of the distal end effector 110 may include greater than 90 degrees of rotation of the distal end effector 110. The operator may effect the rotation, including greater than 90 degrees rotation, of the distal end effector 110 while operating the handle 114 with only one hand. Also, the first operator may move the handle 114 to effect simultaneous unison longitudinal movement of the outer and inner members 106, 108 and/or the distal end effector 110, and axial rotation of the outer and inner members 106, 108 and/or the distal end effector 110.

In addition, the operator may move the first and second members 116, 118 relative to each other to effect relative longitudinal movement of the outer and inner members 106, 108, while also moving the handle 114 as a whole to effect unison longitudinal movement of the outer and inner elongate members 106, 108 and/or to effect axial rotational movement of the outer elongate member 106, the inner elongate member 108, and/or the distal end effector 110. As a particular example, for embodiments where the distal end effector 110 is configured as a clip assembly 304 (FIGS. 3A, 3B), the operator may move the first and second members 116, 118 relative to each other while moving the handle 114 as a whole to effect simultaneous opening or closing of the jaws or legs of the clip assembly 304 while axially rotating the clip assembly 304.

For at least some embodiments such as shown in FIGS. 1A, 1B, 3A-4B, the first member 116 may include a holder 150, such as a clip, that can hold, retain, and/or attach to a portion 152 of the outer elongate member 106 distal the distal end 122 of the first member 116. Correspondingly, proximal portions of the outer and inner elongate members 106, 108 may extend from the distal end 122 of the first member 116 to the holder 150, such as in the form of a loop 154 for example, as shown in FIG. 4. The outer and inner elongate members 106, 108 may then extend from the holder 150 to the endoscope opening 408 and down through the working channel of the endoscope 400.

By attaching to the portion 152 of outer elongate member 106 at the holder 150, the handle 114 exerts distally transferred forces on the outer and inner elongate members 106, 108 at the holder 150 instead of at the distal end 122. Such distally transferred forces may include longitudinal forces that longitudinally move the outer and inner elongate members 106, 108 and the distal end effector 110 in unison with each other, and/or axial rotational forces that axially rotate the outer elongate member 106, the inner elongate member 108, and/or the distal end effector 110. In various embodiments and/or circumstances, the axial rotation may include relative axial rotation where the outer elongate member 106 axially rotates (clockwise or counter-clockwise) corresponding to movement of the first member 116, relative to the inner elongate member 108 and/or the distal end effector 110. In other embodiments, the axial rotation may include unison axial rotation of the outer and inner elongate members 106, 108 and the distal end effector 110.

Accordingly, the holder 150 effectively shortens the length of the outer and inner elongate members 106, 108 between the working channel opening 408 and the device handle 114 compared to the length of the outer and inner elongate members 106, 108 between the working channel opening 408 and the distal end 122. The shortening of the length may enhance the ability of the device handle 114 to effect longitudinal and/or rotational movement of the outer and inner elongate members 106, 108 and/or the distal end effector 110. Accordingly, the holder 150 attaching to the portion 152 of the outer elongate member 106 distal the distal end 122 of the first member 116 may make easier the operator’s control of longitudinal and/or axial rotational movement of the outer and/or inner elongate members 106, 108 and/or the end effector 110 through movement of the handle 114, compared to if the handle 114 instead exerted such longitudinal and/or axial rotational forces on the outer and/or inner elongate members 106, 108 at the distal end 122 of the first member 116.

Handles different than the handle 114 of the present description may similarly be able to effect relative or unison longitudinal movement and/or axial rotational movement of the outer and inner members 106, 108 and/or manipulate the distal end effector 110. However, such handles may require use of two operators, a first operator to hold and operate the endoscope 402 and a second operator to hold and operate the device handle 114. If the first operator tried to hold these other handles while also holding the endoscope 402, the portions of the outer and inner elongate members between the handle and the working channel opening 408 would be too long for effective movement of the outer and inner members 106, 108, and/or the way that the first operator would have to grasp the handle to properly use it while also holding the endoscope 402 would be uncomfortable. In contrast, the handle 114 described herein allows a single operator to comfortably hold and control both the endoscope handle 404 and the device handle 114 while also allowing for relatively unhindered movement of the outer and inner elongate members 106, 108 for performance of the medical procedure for which the medical device 100 is intended.

Additionally, the ability of one operator to simultaneously hold and control the endoscope handle 404 and the device handle 114 may be desirable to the operator since the operator does not have to rely on a second operator to hold and control the device handle 114 during operation of the medical procedure. For these other procedures where the second operator is operating the device handle 114, the first operator holding the endoscope 400 may issue audible commands or instructions to the second operator to carry out the medical procedure. The ability of the first operator operating the endoscope 400 to also hold and operate the device handle 114 may allow for more accurate placement and operation of the distal end effector 110 at the treatment site, compared to other procedures where the second operator is operating the handle 114 in response to audible instructions, resulting in an overall improvement in performance of the medical procedure.

The subject matter of the present description may also relate, among others, to the following aspects:

A first aspect includes a handle for a medical device comprising: a first member from which an inner elongate member and an outer elongate member extend; and a second member configured to move toward and away from the first member, wherein movement of the second member toward and away from the first member controls relative longitudinal movement of the inner and outer elongate members.

A second aspect includes the first aspect, and further includes: wherein the movement of the second member toward and away from the first member comprises rotational movement.

A third aspect includes any of the first or second aspects, and further includes that the first member and the second member are connected to each other via a hinge about which the first member and the second member pivotably move relative to each other.

A fourth aspect includes any of the first through third aspects, and further includes that the second member comprises a bell crank comprising a pair of arms configured to move opposite from each other toward and away from the first member.

A fifth aspect includes the fourth aspect, and further includes that one of the pair of arms is configured to move toward and away from a proximal end of the first member.

A sixth aspect includes the fifth aspect, and further includes that the one of the arms is configured to translate movement toward and away from the proximal end of the first member to longitudinal movement of the inner elongate member.

A seventh aspect includes any of the fourth through sixth aspects, and further includes that one of the pair of arms is configured to move toward and away from an elongate side of the first member.

An eighth aspect includes any of the fourth through seventh aspects, and further includes that the bell crank further comprises an arm connection element connected to each of the pair of arms and circumferentially extending about a pivot point of the bell crank.

A ninth aspect includes the eighth aspect, and further includes that the arm connection element extends and is configured to move through the first member.

A tenth aspect includes the ninth aspect, and further includes that the arm connection element extends through the first member between a pivot point of the bell crank and a proximal end of an elongate body of the first member.

An eleventh aspect includes any of the fourth through tenth aspects, and further includes that the pair of arms forms an angle greater than 90 degrees and less than 180 degrees.

A twelfth aspect includes any of the first through eleventh aspects, and further includes that the second member is configured to move toward the first member to configure the handle in a closed state and move away from the first member to configure the handle in an open state.

A thirteenth aspect includes the twelfth aspect, and further includes that the first member and the second member form an angle that is less than 90 degrees in the open state.

A fourteenth aspect includes any of the first through thirteenth aspects, and further includes that the second member comprises a finger-securing portion disposed on a surface of the second member that faces in a direction that the second member moves away from the first member.

A fifteenth aspect includes any of the first through fourteenth aspects, and further includes that the first member comprises a holder configured to hold a portion of the outer elongate member.

A sixteenth aspect includes a medical device comprising: an outer elongate member; an inner elongate member movably disposed within the outer elongate member, each of the outer elongate member and the inner elongate member longitudinally extending from a proximal portion to a distal portion; a distal end effector coupled to a distal end of the elongate inner member; and a handle coupled to the proximal portion, the handle comprising a first member and a second member, wherein the outer and inner elongate members project from a distal end of the first member, and wherein the first and second members are configured to pivot relative to each other to longitudinally move the outer and inner elongate members relative to each other.

A seventeenth aspect includes the sixteenth aspect, and further includes that the first member and the second member are connected to each other via a hinge about which the first member and the second member pivot relative to each other.

An eighteenth aspect includes the seventeenth aspect, and further includes that the second member comprises a bell crank integrated with the hinge.

A nineteenth aspect includes any of the sixteenth through eighteenth aspects, and further includes that the first member comprises an elongate body, and a holder configured to hold a portion of the outer elongate member, wherein the outer and inner elongate members form a loop between a distal end of the elongate body and the holder.

A twentieth aspect includes any of the sixteenth through nineteenth aspects, and further includes that the distal end effector comprises a clip assembly.

A twenty-first aspect includes method of operating a medical device, the method comprising: positioning a handle in an open state, wherein the handle comprises a first member and a second member pivotably connected to each other, wherein outer and inner elongate members of the medical device distally extend from a distal end of the first member, wherein, in the open state, the first and second members form an angle less than 90 degrees; rotating the second member toward the first member; and proximally moving the inner elongate member relative to the outer elongate member in response to rotating the second member toward the first member.

A twenty-second aspect includes the twenty-first aspect, and further includes that the second member comprises a bell crank comprising a first arm and a second arm, and wherein rotating the second member toward the first member comprises: rotating the first arm toward an elongate side of the first member and rotating the second arm away from a proximal end of the first member.

A twenty-third aspect includes any of the twenty-first or twenty-second aspects, and further includes: deploying a clip assembly at a treatment site in response to moving the handle from the open state to a closed state.

A twenty-fourth aspect includes any of the twenty-first through twenty-third aspects, and further includes: rotating the second member away from the first member; and distally moving the inner elongate member relative to the outer elongate member in response to rotating the second member away from the first member.

A twenty-fifth aspect includes any of the twenty-first through twenty-fourth aspects, and further includes: with a holder of the first member, exerting an axial rotational force on the outer elongate member to effect axial rotation of at least one of the outer elongate member, the inner elongate member, or a distal end effector.

A twenty-sixth aspect includes the twenty-fifth aspect, and further includes: axially rotating the distal end effector more than 90 degrees in response to exerting the axial rotational force.

A twenty-seventh aspect includes any of the twenty-first through twenty-sixth aspects, and further includes: simultaneously exerting the axial rotational force on the outer elongate member and rotating the second member toward or away from the first member.

A twenty-eighth aspect includes any of the twenty-first through twenty-seventh aspects, and further includes: simultaneously moving the handle toward or away from a proximal working channel opening and rotating the second member toward or away from the first member.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A handle for a medical device, the handle comprising:
a first member from which an inner elongate member and an outer elongate member extend; and
a second member configured to move toward and away from the first member,
wherein movement of the second member toward and away from the first member controls relative longitudinal movement of the inner and outer elongate members.

2. The handle of claim 1, wherein the movement of the second member toward and away from the first member comprises rotational movement.

3. The handle of claim 1, wherein the first member and the second member are connected to each other via a hinge about which the first member and the second member pivotably move relative to each other.

4. The handle of claim 1, wherein the second member comprises a bell crank comprising a pair of arms configured to move opposite from each other toward and away from the first member.

5. The handle of claim 4, wherein one of the pair of arms is configured to move toward and away from a proximal end of the first member.

6. The handle of claim 5, wherein the one of the arms is configured to translate movement toward and away from the proximal end of the first member to longitudinal movement of the inner elongate member.

7. The handle of claim 4, wherein one of the pair of arms is configured to move toward and away from an elongate side of the first member.

8. The handle of claim 4, wherein the bell crank further comprises an arm connection element connected to each of the pair of arms and circumferentially extending about a pivot point of the bell crank.

9. The handle of claim 8, wherein the arm connection element extends and is configured to move through the first member.

10. The handle of claim 9, wherein the arm connection element extends through the first member between a pivot point of the bell crank and a proximal end of an elongate body of the first member.

11. The handle of claim 4, wherein the pair of arms forms an angle greater than 90 degrees and less than 180 degrees.

12. The handle of claim 1, wherein the second member is configured to move toward the first member to configure the handle in a closed state and move away from the first member to configure the handle in an open state.

13. The handle of claim 12, wherein the first member and the second member form an angle that is less than 90 degrees in the open state.

14. The handle of claim 1, wherein the second member comprises a finger-securing portion disposed on a surface of the second member that faces in a direction that the second member moves away from the first member.

15. The handle of claim 1, wherein the first member comprises a holder configured to hold a portion of the outer elongate member.

16. A medical device comprising:
an outer elongate member;
an inner elongate member movably disposed within the outer elongate member, each of the outer elongate member and the inner elongate member longitudinally extending from a proximal portion to a distal portion;
a distal end effector coupled to a distal end of the elongate inner member; and
a handle coupled to the proximal portion, the handle comprising a first member and a second member,
wherein the outer and inner elongate members project from a distal end of the first member, and
wherein the first and second members are configured to pivot relative to each other to longitudinally move the outer and inner elongate members relative to each other.

17. The medical device of claim 16, wherein the first member and the second member are connected to each other via a hinge about which the first member and the second member pivot relative to each other.

18. The medical device of claim 17, wherein the second member comprises a bell crank integrated with the hinge.

19. The medical device of claim 16, wherein the first member comprises an elongate body, and a holder configured to hold a portion of the outer elongate member, wherein the outer and inner elongate members form a loop between a distal end of the elongate body and the holder.

20. The medical device of claim 16, wherein the distal end effector comprises a clip assembly.
